(19) United States
(12) Patent Application Publication
(10) Pub. No.: US 2012/0143045 A1
(43) Pub. Date: Jun. 7, 2012

(54) METHOD FOR IMAGE SUPPORT IN THE NAVIGATION OF A MEDICAL INSTRUMENT AND MEDICAL EXAMINATION DEVICE

(76) Inventor: Klaus Klingenbeck, Aufsess (DE)

(21) Appl. No.: 13/306,169

(22) Filed: Nov. 29, 2011

(30) Foreign Application Priority Data
Dec. 2, 2010 (DE) 102010062340.7

(51) Int. Cl. A61B 5/055

(52) U.S. Cl. 600/424

(57) ABSTRACT

A method for image support in a navigation of a medical instrument, in particular a catheter, in at least one hollow organ in a surgical site of a body is proposed. A presentation of a current position of the instrument in the hollow organ is generated from a three-dimensional dataset of the surgical site and the presentation data describes the current position of the instrument. At least one geometry parameter influencing the generation and/or display of the presentation is automatically adjusted taking into account position data of the instrument describing the current three-dimensional position and the current three-dimensional orientation of a tip of the instrument. The presentation corresponding to the geometry parameters is displayed.

### CROSS REFERENCE TO RELATED APPLICATIONS

This application claims priority of German application No. 10 2010 062 340.7 filed Dec. 2, 2010, which is incorporated by reference herein in its entirety.

### FIELD OF THE INVENTION

The invention relates to a method for image support in the navigation of a medical instrument, in particular a catheter, in at least one hollow organ in a surgical site of a body, wherein a presentation of the current position of the instrument in the hollow organ is generated from a three-dimensional dataset of the surgical site and presentation data describing the current position of the instrument, as well as a medical examination device for implementation of the method.

### BACKGROUND OF THE INVENTION

In minimally invasive surgery a medical instrument, for example a catheter or an endoscope, is used for navigation in a hollow organ of a patient, in particular the blood vessels or the heart. An example of a procedure of this type is the insertion of ablation catheters into heart ventricles, for example to treat atrial fibrillation in the left atrium.

To be able to actually guide the instrument to the right destination in order to carry out the treatment there, methods for image support in the navigation of the instrument have been proposed in which the position of the instrument, mostly specifically the tip of the instrument, is to be visualized in a three-dimensional presentation of the hollow organ. To this end a three-dimensional dataset of the surgical site is used which clearly shows the at least one hollow organ through which the instrument is to be navigated. The three-dimensional dataset can here be obtained from at least one image dataset or an image dataset can be used directly, for example an MR dataset, a CT dataset or the like. For the most part contrast-medium-enhanced 3D recordings are produced for this, in which the relevant hollow organ is segmented using known methods. The result of the segmentation is a three-dimensional mapping of the inner surface of the organ, for example of the endocardium of the atrium.

The realtime control of the navigation of the instrument is normally achieved by recording radioscopy images (fluoroscopy images), in other words two-dimensional X-ray images, whereby by means of a 2D/3D registration the instrument can be visualized geometrically precisely in three-dimensional space together with the three-dimensional dataset. Although it is also known for the position of the instrument to be determined using a position determination system, which for example works on the basis of sensors, nevertheless it is for the most part preferred to record two-dimensional fluoroscopic images, since the three-dimensional data of the dataset is static and the assignment of the position may be imprecise, in particular when work is to be performed in rapidly moving surgical sites, for example on the heart. In two-dimensional fluoroscopic images the movement itself can be seen. Ultrasound images are sometimes used as an alternative to fluoroscopic recordings.

As already mentioned, it is known for a presentation of the current position and orientation of the instrument in the hollow organ to be generated, in that ultimately the three-dimensional dataset of the surgical site and the presentation data that describes the position and orientation of the instrument, in particular of the tip of the instrument, wherein the three-dimensional dataset and the presentation data are registered with one another, are merged. The presentation can then be displayed on a display device, for example a monitor, to the person performing the surgery.

However, the problem with this is that it can happen that in the three-dimensional datasets known structures can overlap the mapping of the instrument in the presentation. For example, the tip of a catheter can be located on the rear wall of the atrium, but is overlaid by the front wall of the atrium such that the catheter superimposed onto the presentation is no longer visible. To solve this problem it has been proposed to increase the transparency of the anatomical structures shown in the presentation, which however taken as a whole results in poorer recognizability of the overall presentation.

Hence it is generally preferred to set “clip planes” which define regions of the three-dimensional dataset not to be taken over into the presentation. In this way it is possible to look into the hollow organ without any obstruction and to track the instrument in the hollow organ. The problem with this is that the position and orientation of the clip plane must be set manually by the person performing the surgery or by an assistant. With every significant movement of the instrument this setting has to be performed or optimized afresh. This interaction of users is particularly cumbersome in the sterile environment of a catheter laboratory or other areas for minimally invasive surgery.

EP 2 147 636 A1 describes an apparatus and a method for guiding surgical tools using ultrasound imaging. This aims to create a pure realtime method which does not require any previously recorded images. Consequently it is there proposed to record a time sequence of three-dimensional ultrasound images in real time, with the position and orientation of a surgical instrument being likewise tracked in real time, so that a characteristic axis of the tool can be defined, this mainly being geared to application in the field of needles, canulas, etc. inserted through the skin.

Since the relative position in space between the three-dimensional ultrasound image and the characteristic axis of the tool has now been determined, a realtime 2D image can now be generated which is defined by an image plane through the corresponding three-dimensional image, and consequently represents a sectional image. This sectional image should now be recorded so that all pixels at a defined distance from the tip of the tool are displayed to someone sitting on said tip of the tool, if this person is looking down from said tip. The clip plane can also be selected in parallel to the characteristic axis of the tool, but in this case too does not show the tool, but merely parallel lines that show the extension of the tool along the characteristic axis.

### SUMMARY OF THE INVENTION

The object of the invention is hence to improve image monitoring of a medical instrument, in line with the situation, in respect of manageability, legibility and the information contained therein.

To achieve this object it is inventively provided in a method that at least one geometry parameter influencing the generation and/or display be automatically adjusted, taking into account position data of the instrument describing the current three-dimensional position and the current three-dimensional orientation of a tip of the instrument and that the presentation corresponding to the geometry parameters be displayed.

The presentation is inventively thus adjusted completely automatically as a function of current position data of the instrument, whereby in the context of this description “position” is also to be understood in the following as the six-dimensional position, in other words the position and orientation. Preferably it can be provided here that the viewing direction of the presentation and/or at least one clip plane defining regions of the three-dimensional dataset not to be taken over into the presentation are used as geometry parameters. As regards the viewing direction, this can ultimately always be selected so that a good view is obtained of the feed motion of the instrument, in particular of the catheter. For example, a basic viewing direction relative to the instrument can be defined for this, for example a view from obliquely behind in the direction of feed. The viewing direction, shown in the presentation, to the hollow organ in which the instrument is located is then always adjusted as a function of the position data, and is consequently advantageously updated in real time. The person performing the surgery or an assistant then no longer needs to carry out any other operator functions. This is extremely advantageous in the sterile region in particular.

Furthermore a clip plane can always be maintained as a function of the position data so that a view of the instrument is possible. Preferably an unobstructed view is additionally in principle available in the direction of a target position or the target position itself. In a particularly advantageous embodiment the viewing direction and the clip plane are jointly adjusted in real time and thus are kept updated in line with the movement of the instrument, so that not only is there an optimum viewing direction for the further feed motion of the instrument, but in addition it is always ensured that an unobstructed view of the instrument exists, in particular, without thereby masking out the target position.

In a concrete embodiment it can here be provided that a clip plane to be adjusted on the basis of the position data is defined at a fixed distance and a fixed angle of inclination to the tip of the instrument, in particular automatically or semi-automatically. Analogously, as already described, it can be provided that the viewing direction is adjusted on the basis of the position data, in particular relative to the orientation of the tip of the instrument, wherein this too is preferably possible automatically and/or at least at the start of the surgery. As regards the clip plane, it can here particularly advantageously be provided that the definition is effected as a function of at least one target position marked in particular in the three-dimensional dataset and/or a set viewing direction. This means it is on the one hand possible, for a target position, for example a location to be treated, to be marked beforehand, for example by a user, which target position can likewise be taken into account when setting the clip plane (and also, as addressed in greater detail in the following, the viewing direction). For example, the target position can be used when adjusting the geometry parameters such that the clip plane is selected so that the target position still remains visible. But the definition of the clip plane can also be effected as a function of the viewing direction, since ultimately this specifies in what regions of the three-dimensional dataset structures overlaying the instrument may potentially exist which should be cropped. It is also particularly advantageous here if both the at least one target position and the set viewing direction are taken into account.

It is also conceivable for the definition to be effected as a function of a user input. However, at least during surgery this should only be necessary in exceptional cases, for example if the person performing the surgery has “misnavigated”, in particular, in that a target position now lies behind the instrument or similar. The geometry parameters of the presentation may then need to be completely reset manually, which advantageously can be effected on a graphical user interface. In this connection it can be provided in an advantageous embodiment that to assist with the user input a schematic presentation of the instrument can in particular be presented. For example, the clip plane can be presented at the same time as the instrument, so that a user can grip, move and/or tilt it using a suitable tool. Preferably the viewing direction to the schematic presentation of the instrument and the clip plane is selected such that it corresponds to the viewing direction currently set for the up-to-date presentation.

As regards the viewing direction, it can be expediently provided that it is selected by taking account of a straight line connecting the tip of the instrument to a target position marked in particular in the three-dimensional dataset, in particular along the straight line. In this way the person performing the surgery can be made intuitively aware of the direction in which the current target position is located, so that he or she can navigate particularly purposefully to the target position. Alternatively it is obviously also conceivable for the viewing direction to be selected as a function of the orientation of the tip of the instrument, in particular in the direction of slide of the instrument or in a fixed angular position thereto. Thus the regions of the hollow organ lying in front of the instrument are always in the view of the person performing the surgery. It is also conceivable for a user interface to be used to toggle between the way in which the geometry parameters, in particular the viewing direction, are determined.

It is generally advantageous in this connection if both the viewing direction and the clip plane are automatically continuously updated as geometry parameters, in particular in real time, if the new viewing direction is always determined first and if the clip plane then is correspondingly updated taking account of this new viewing direction set. In this way the best possible presentation for the person performing the surgery is always achieved.

Preferably the position data is determined using at least one position sensor arranged on the instrument, in particular an electromagnetic position sensor. Thus an instrument is used which for example comprises at least one position sensor provided in or on the tip of the instrument. A position sensor of this type, in particular an electromagnetic position sensor, can then determine the spatial coordinates of the tip of the instrument and its direction angle in space as position data. Such position determination systems and their registration with image recording modalities or similar are widely known in the prior art and need not be explained further here.

Alternatively it is in principle also conceivable for fluoroscopic images recorded for example at an angle, in particular 90°, to one another to be used to determine the position data. However, this is less preferred, since fluoroscopic images from different angles can only with difficulty be recorded on an up-to-date basis. If a biplane X-ray device is used, space problems can occur.

It can further be provided that at least some of the position data corresponds to the presentation data. For reasons mentioned in the introduction it is however preferably the case that a current fluoroscopic image of the surgical site is used as at least a part of the presentation data. The tip of the instrument is for the most part readily recognizable in this.

As already mentioned in the introduction, fluoroscopy monitoring is in principle expedient in respect of the traceability of movements in the surgical site, so that a position determination system is preferably provided in parallel with fluoroscopy monitoring. In this case the data can obviously be used in common, providing mutual plausibility if necessary, wherein data of the position determination system can in addition supply information on the missing spatial direction in the fluoroscopic images, which are in fact two-dimensional. Even where in the following only the data of the position determination system is used as position data, some of the position data is nonetheless also included as presentation data.

The three-dimensional dataset can be an image dataset recorded beforehand of the surgery area and/or a dataset derived from such an image dataset. For example, the three-dimensional dataset may be based on a magnetic resonance image dataset, a computed tomography image dataset and/or a three-dimensional image dataset recorded using another modality, which then for example is further processed using segmentation methods known in the prior art, in order to extract the inner surface of the hollow organ in which navigation is effected and to create a model of the hollow organ for example as a three-dimensional dataset, in which the instrument is then navigated. For example, in this way a model of the heart and of the surrounding blood vessels can be generated if this corresponds to the surgical site.

Besides the method the present invention also relates to a medical examination device, comprising a display device and a control device designed for implementing the inventive method. All explanations relating to the inventive method can be transferred analogously to the inventive examination device, so that the advantages of the invention can also be achieved herewith. An inventive examination device can for example comprise an X-ray device with a C-arm, on which an X-ray tube and an X-ray receiver are arranged opposite one another. This can be used to record fluoroscopic images as presentation data or as a basis for the presentation data. At the same time a medical instrument can be provided which contains position sensors built into its tip, which are part of an in particular electromagnetic position determination system. A three-dimensional dataset can be obtained via a corresponding communication link, and forms the basis for the presentation to be generated, wherein it is advantageously also conceivable for a three-dimensional image dataset to be generated with the X-ray device also used for recording fluoroscopic images, for example, in that during the rotation of the C-arm projection images are recorded at different projection angles and from these a three-dimensional image dataset is generated in known fashion. If necessary a contrast medium can be administered here beforehand. A three-dimensional image dataset of this type, recorded using a C-arm-X-ray device, has the advantage that even three-dimensional datasets derived therefrom, which for example are obtained by corresponding segmentation, can already be registered with the fluoroscopic images, in particular, if the patient remains motionless. If the position determination system is moreover permanently integrated into the medical examination device, then a fixed registration can also exist in respect of the position determination system and the X-ray device. Thus an environment is created which is excellently suited for implementation of the inventive method.

### BRIEF DESCRIPTION OF THE DRAWINGS

Further advantages and details of the present invention emerge from the exemplary embodiments described in the following as well as on the basis of the drawing, in which:

FIG. 1 shows an inventive examination device,

FIG. 2 shows a sketch in explanation of the inventive method and

FIG. 3 shows a possible user interface for setting a relative position of a clip plane.

### DETAILED DESCRIPTION OF THE INVENTION

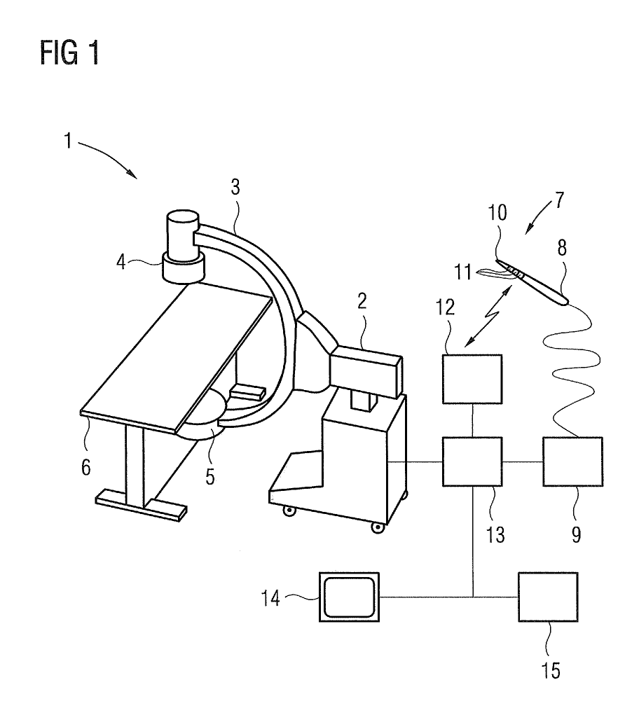

FIG. 1 shows an inventive medical examination device 1. It comprises an X-ray device 2 with a C-arm 3, on which an X-ray tube 4 and an X-ray receiver 5 are arranged opposite one another. The C-arm 3 can here be moved in respect of at least one degree of freedom of movement, in particular one degree of freedom of rotation, relative to a patient couch 6.

Furthermore, a catheter 8, here an ablation catheter, is provided as a medical instrument 7 to be inserted into a hollow organ for treatment, and is connected to a catheter control device 9. Electromagnetic position sensors 11 are provided in the tip 10 of the catheter 8 as in principle known, and are assigned to a position determination system 12 which for example can generate an external magnetic field, in order to measure signals induced in the position sensors 11 and from them to determine the six-dimensional orientation of the tip of the instrument 10, in other words the three-dimensional position and the three-dimensional orientation of the tip of the instrument 10.

The X-ray device 2, the position determination system 12 and the catheter control device 9 are connected to a control device 13 which controls the operation of the medical examination device 1 and is designed for implementing the inventive method, which is explained in greater detail in the following.

The control device 13 further has access to a display device 14, here a monitor, and an operator device 15.

The control device 13 is now able, by taking account of position data, to automatically adjust geometry parameters of a three-dimensional presentation showing the hollow organ in the surgical site with the current position of the catheter 8, in particular the tip of the instrument 10, said presentation being obtained from a three-dimensional dataset and presentation data describing the position of the catheter 8, if the catheter 8 is moved, in other words changes its position. The viewing direction and the position of a clip plane, which defines regions of the three-dimensional dataset not to be taken over into the presentation, are automatically adjusted here.

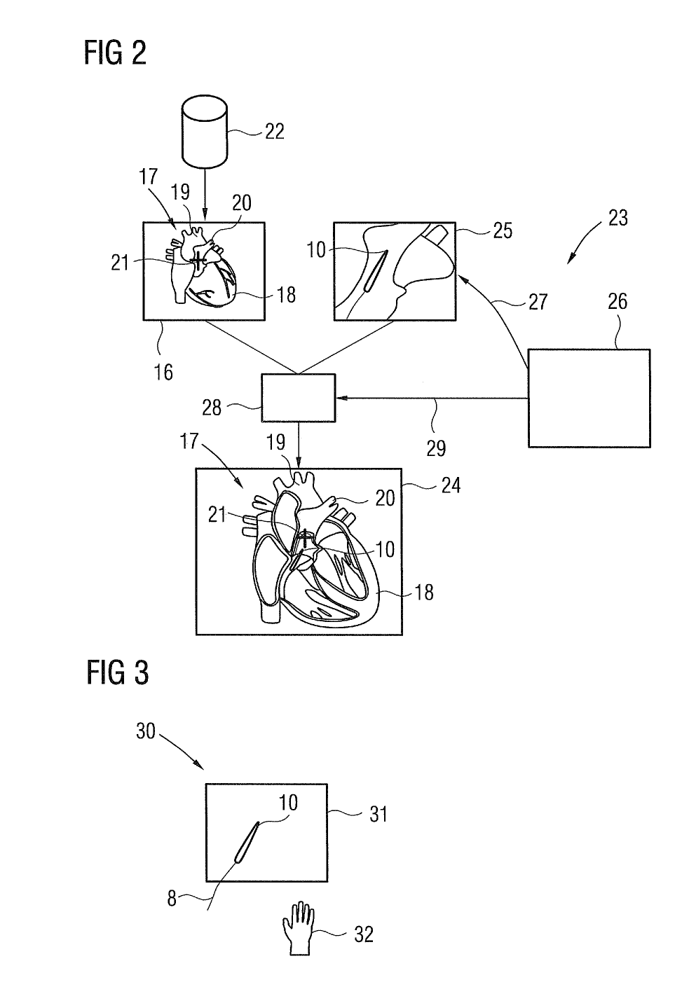

This will now be explained in greater detail with the aid of FIG. 2. The method is based, as described, on a three-dimensional dataset 16 of the surgical site 17, which shows particularly clearly or even exclusively the inner walls of the hollow organs to be traversed, in the exemplary embodiment according to FIG. 2 for example the heart 18 with the surrounding blood vessels 19, in particular the pulmonary vein 20, which in this instance contains the destination 21 of the surgery.

In this example the three-dimensional dataset 16 is obtained from a three-dimensional image dataset 22 which was recorded using the X-ray device 2. To this end a plurality of projection images was recorded from different angles during a rotation of the C-arm, and was transferred to the three-dimensional image dataset 22 using a reconstruction method. Since a contrast medium was administered prior to recording this three-dimensional image dataset 22, the heart 18 and the blood vessels 19 can be recognized particularly clearly. Hence the heart 18 and the blood vessels 19 can be segmented using a standard segmentation method, so that finally the inner boundaries of the heart 18 and of the blood vessels 19 can be used as a basis for the three-dimensional dataset 16, which ultimately represents a model which contains the hollow organs in their position.

The three-dimensional dataset 16 can be used prior to the planned minimally invasive surgery with the catheter 8, in order to plan the surgery, which means the destination 21 can be marked in the three-dimensional dataset 16.

The aim is now to use the three-dimensional dataset 16 jointly with presentation data 23 in order to generate a three-dimensional presentation 24 that shows both the anatomy of the surgical site 17 and the current position of the catheter 8. Two-dimensional fluoroscopic images 25 from the X-ray device 2, recorded at regular intervals, and from which the tip of the instrument 10 is readily apparent, are here used as presentation data. This position information is supported by position data 26 obtained from the position determination system 12, cf. arrow 27.

However, on looking at the three-dimensional dataset 16 it is clear that a catheter 8 moving inside the hollow organs 18, 19 is not visible at all, since the front walls may cover the catheter 8. Consequently two essential geometry parameters 28 exist which also influence the optimum legibility and utility of the three-dimensional presentation 24, namely on the one hand the viewing direction from which the scene is viewed, but on the other hand also at least one clip plane which determines which regions of the three-dimensional dataset 16 should not be visible in the presentation 24, in order that the catheter 8 (and if necessary the destination 21) are visible.

In the inventive method the position data 26 from the position determination system 12 is hence now used in order to update the geometry parameters 28 automatically in the case of updated position data 26, arrow 29. In this instance, if new position data 26 exists, the viewing direction is first adjusted as geometry parameters 28 to the new position of the catheter 8, in particular the tip of the instrument 10. This happens in this instance in that a connection line is drawn from the tip of the instrument 10 to the destination 21 and on the basis of this connection line the viewing direction is defined, for example so that a user, when the presentation 24 is displayed on the display device 14, has a good view of both the catheter 8 and the destination 21, in other words ultimately also the path to the destination 21, for example in the form of an oblique top view. To this end a fixed angle of inclination of the viewing direction to the connection line can for example be used. Alternatively it is also possible that the current orientation of the tip of the catheter, in other words the direction of feed motion of the catheter 8, acts as a reference for the definition of the viewing direction. It may be noted that more complex possibilities for determining a viewing direction from the position data 26 are obviously also possible, which for example seek to achieve a good view of the hollow organ 18, 19 lying in front of the catheter 8 and the destination 21. It is possible to toggle between different possibilities for automatically setting the viewing direction, for example using the operator device 15.

If the viewing direction is first known, the clip plane can also be updated. It is here possible that the position of the clip plane likewise orients itself to the viewing direction, but it is also conceivable for the clip plane to be defined essentially at a fixed distance and at a fixed angle to the position and orientation of the tip of the instrument 10. Thus the clip plane too is kept updated—be it directly or indirectly—as a function of the position data 26.

The result is a presentation 24, as shown for example in FIG. 2. It can be seen that because of the clip plane a part of the heart 18 and of the blood vessels 19 is now shown unobstructed, in particular also the pulmonary artery 20. The tip of the instrument 10 and the destination 21 are readily recognizable, as is the path on which the destination 21 can be achieved.

Since the steps of the inventive method are executed completely automatically, no operator interaction is necessary in order to maintain a constant presentation of an up-to-date and optimally legible image.

If nonetheless a change in the basic parameters set for the automatic updating of viewing direction and clip plane should be necessary, for example, if the destination 21 has mistakenly already been passed by the catheter tip 10 or similar, the user interface 30 shown in FIG. 3 can for example be used to redefine the parameters for determining the clip plane. The catheter 8 with the tip of the instrument 10 is shown schematically there. The clip plane 31 is also shown in the schematic three-dimensional presentation relative to the catheter, and can be gripped and correspondingly manipulated, in particular moved or rotated, using a corresponding tool, in this case a gripper hand 32. The whole presentation can also be changed. A similar possibility for setting is also conceivable in respect of the viewing direction.

1. A method for image support in a navigation of a medical instrument in a hollow organ in a surgical site of a body, comprising:
generating a three-dimensional presentation data of the instrument at a current position in the hollow organ from a three-dimensional dataset of the surgical site;
automatically adjusting a geometry parameter influencing the generation and/or display of the presentation data based on position data of the instrument describing a current three-dimensional position and a current three-dimensional orientation of a tip of the instrument; and
displaying the presentation data corresponding to the geometry parameter,
wherein the geometry parameter comprises a viewing direction of the presentation data and/or a clip plane defining a region of the three-dimensional dataset that are not to be taken into the presentation data.

2. The method as claimed in claim 1, wherein the clip plane is defined at a fixed distance and a fixed angle of an inclination to the tip of the instrument.

3. The method as claimed in claim 2, wherein the clip plane is defined automatically or semi-automatically.

4. The method as claimed in claim 2, wherein the clip plane is defined as a function of a target position marked in the three-dimensional dataset and/or of the viewing direction.

5. The method as claimed in claim 2, wherein the clip plane is defined by an input of a user input.

6. The method as claimed in claim 5, wherein a schematic presentation of the instrument is presented to the user for making the input.

7. The method as claimed in claim 1, wherein the viewing direction is selected based on a straight line connecting the tip of the instrument to a target position marked in the three-dimensional dataset.

8. The method as claimed in claim 1, wherein the viewing direction is selected based on the orientation of the tip of the instrument.

9. The method as claimed in claim 1, wherein the orientation of the tip of the instrument is a direction for sliding the instrument.

10. The method as claimed in claim 1, wherein the position data is determined using at least one position sensor arranged on the instrument.

11. The method as claimed in claim 10, wherein the position sensor is an electromagnetic position sensor.

12. The method as claimed in claim 1, wherein the presentation data comprises at least some of the position data.

13. The method as claimed in claim 1, wherein the presentation data comprises a current fluoroscopic image of the surgical site.

14. The method as claimed in claim 1, wherein the three-dimensional dataset comprises an image recording dataset of the surgery site and/or a dataset derived from the image recording dataset.

15. A medical examination device for navigating a medical instrument in a hollow organ in a surgical site of a body, comprising:
a control device that is configure to:
generate a three-dimensional presentation data of the instrument at a current position in the hollow organ from a three-dimensional dataset of the surgical site,
automatically adjusting a geometry parameter influencing the generation and/or display of the presentation data based on position data of the instrument describing a current three-dimensional position and a current three-dimensional orientation of a tip of the instrument; and
a display device that displays the presentation data corresponding to the geometry parameter,
wherein the geometry parameter comprises a viewing direction of the presentation data and/or a clip plane defining a region of the three-dimensional dataset that are not to be taken into the presentation data.
